(19) United States
(12) Patent Application Publication
(10) Pub. No.: US 2026/0182859 A1
(43) Pub. Date: Jul. 2, 2026

(54) METHOD AND APPARATUS FOR DETERMINING ARTERIAL CO2 CONCENTRATION

(71) Applicant: LUMETRY DIAGNOSTICS GMBH, Graz (AT)

(72) Inventors: Christian NEUBAUER, Graz (AT); Horst RÜTHER, Hart bei Graz (AT); Max SORANTIN, Hart bei Graz (AT)

(21) Appl. No.: 19/131,386

(22) PCT Filed: Nov. 21, 2023
(86) PCT No.: PCT/EP2023/082452

§ 371 Date: May 20, 2025

(30) Foreign Application Priority Data
Nov. 21, 2022 (EP) 22208583.9

(51) Int. Cl. A61B 5/083; A61B 5/087; A61B 5/097; A61B 5/145; G01N 33/497

(52) U.S. Cl. CPC A61B 5/0836; A61B 5/087; A61B 5/097; A61B 5/14542; G01N 33/497; A61B 2560/0238

(57) ABSTRACT

The present invention provides a method for determining an arterial CO₂ concentration (PaCO₂) level in a subject, the method comprising the following steps: —measuring CO₂ concentration levels in exhaled breath from the subject over a period of time in order to obtain a capnography signal; —deriving a CO₂ waveform signal from the capnography signal; determining an End-tidal CO₂ (EtCO₂) value from the CO₂ wave-form signal; —analyzing a shape of the CO₂ waveform signal in order to determine a CO₂ mismatch correction factor; —applying the CO₂ mismatch correction factor to the EtCO₂ value in order to obtain the PaCO₂ level. The present invention further provides an apparatus for determining a PaCO₂ concentration level.

The field of the present invention is the field of arterial carbon dioxide (CO₂) concentration measurement.

In clinical diagnostics, arterial blood gas analysis measures the amounts of arterial gases in a blood sample taken from a subject's artery. Such analyses are used in various areas of medicine, e.g. emergency medicine or pulmonology. Typically, arterial blood gas analysis includes measurements of the concentration of oxygen (arterial partial pressure of oxygen; PaO₂) and the concentration CO₂ (arterial partial pressure of CO₂; PaCO₂).

PaCO₂ levels are of particular medical interest. For instance, in patients with hypercapnia, the arterial CO₂ partial pressure can reach critical values within just a few days, requiring immediate clinical admission. Since arterial blood gas measurements are not readily available at patients' homes, there is an urgent need for easily accessible low-cost tools to determine PaCO₂ levels without trained personnel.

Respiratory gas analysis has the potential to serve as such a tool. Capnometers and Capnographs measure the concentration of CO₂ in expired air. They typically use infrared measurement to detect the torsional and/or stretching vibration of the CO₂ molecule in order to measure the CO₂ concentration. “Capnometry” is typically used as a general term to refer to the measurement of CO₂ concentrations in respiratory gas. The term “Capnography” typically refers more specifically to the continuous analysis and recording of the CO₂ concentration over a certain period of time. Capnographs typically present the measured data as a series of waveforms representing the CO₂ concentration in the breath as a function of time, e.g., on a display integrated in the capnograph device.

Capnographs are used to assess a patient's respiratory status. Typically, the so-called end-tidal carbon dioxide (EtCO₂) is measured, which is the level of CO₂ at the end of an exhaled breath.

The EtCO₂ (end-tidal CO₂ in exhaled breath) and the PaCO₂ (arterial CO₂) are related, but not the same. The difference between EtCO₂ and PaCO₂ is commonly referred to as “mismatch”. Typical mismatch values are known; thus, it is to some extent possible to draw conclusions about arterial blood partial pressures from capnometric measurements. However, such estimation of PaCO₂ from capnometric measurements lacks accuracy and reliability. This is in particular true for individuals with changes in lung tissue and/or emphysema, where mismatch values can be entirely different from healthy individuals (but this mismatch can only be used as a diagnostic parameter, if PaCO₂ values from blood are available). In many cases capnometric measurements in sick individuals will significantly under- or overestimate PaCO₂ levels, thereby giving the patient a false sense of security.

Methods involving capnographic measurements are known, e.g., from US 2021/244900 A1, U.S. Pat. No. 5,632,281 A, US 2009/118633 A1, Rasera et al (Measurement 44.1 (2011): 60-64), US 2016/150997 A1, Jaffe (Journal of Clinical Monitoring and Computing 31.1 (2017): 19-41) and EP 2 438 855 A2.

Thus, easily accessible yet reliable tools for determining PaCO₂ levels, in particular determining PaCO₂ levels from the breath, especially in sick individuals, are still lacking. It is an object of the invention to provide such tools.

This object is solved by the method and apparatus as defined in the independent claims.

In a first aspect, the invention provides a method for determining an arterial CO₂ concentration (PaCO₂) level in a subject, the method comprising the following steps:
measuring CO₂ concentration levels in exhaled breath from the subject over a period of time in order to obtain a capnography signal;
deriving a CO₂ waveform signal from the capnography signal;
determining an End-tidal CO₂ (EtCO₂) value from the CO₂ waveform signal;
analyzing a shape of the CO₂ waveform signal in order to determine a CO₂ mismatch correction factor;
applying the CO₂ mismatch correction factor to the EtCO₂ value in order to obtain the PaCO₂ level.

In a further aspect, the invention provides an apparatus for determining a PaCO₂ concentration level in a subject, the apparatus comprising:
at least one capnography sensor configured to measure CO₂ concentration levels in exhaled breath from the subject as a function of time;
a processor configured to carry out the following steps:
   obtaining from the capnography sensor a capnography signal comprising CO₂ concentration levels in exhaled breath from the subject over a period of time;
   deriving a CO₂ waveform signal from the capnography signal;
   determining an End-tidal CO₂ (EtCO₂) value from the CO₂ waveform signal;
   analyzing a shape of the CO₂ waveform signal in order to determine a CO₂ mismatch correction factor;
   applying the CO₂ mismatch correction factor to the EtCO₂ value in order to obtain the PaCO₂ level.

Preferably, the processor of the inventive apparatus is configured to carry out the steps as defined with respect to the inventive method. Thus, herein all detailed descriptions of the inventive method equally apply to the apparatus of the invention and vice versa.

The invention is based on the surprising finding, that the mismatch between the arterial PaCO₂ value and the respiratory EtCO₂ is correlated to the shape of the waveform of CO₂ concentration in exhaled air. Thus, it was surprisingly found that by analyzing the shape of a capnographic curve, it is possible to estimate the mismatch and thus the PaCO₂ level. This is of special importance for subjects with medical conditions, for whom the mismatch is often particularly large. However, also for healthy subjects, the inventive method allows determining PaCO₂ more precisely.

Already prior to the invention it was known that certain disease states can influence the shape of a capnographic curve. For instance, EP 2 438 855 A2 relates to methods for interpreting capnographic waveforms. A first aspect disclosed therein relates to a method for diagnosis of a respiratory status of a patient, comprising analyzing a shape of a recorded CO₂ waveform; extracting, from the analyzed shape, at least one parameter by which the shape is characterized; and generating a diagnosis of the respiratory status of the patient, based on the at least one parameter. The parameter, on which the diagnosis is based, may e.g. be one or more of an angle (α) between an alveolar rise and an alveolar plateau of the waveform, an angle (β) between the alveolar plateau and a descending limb of the waveform, EtCO₂, a final inspired CO₂ level (FiCO₂), or an overall rate of rise of CO₂. For instance, with respect to the angle α, EP 2 438 855 A2 teaches that said angle is determined primarily by the V/Q (ventilation/perfusion) status of the lungs, and that patients with obstructions of the airway, such as in the case of chronic obstructive pulmonary disease (COPD) or asthma, have an increased a angle. According to EP 2 438 855 A2, the a angle is thus a widely used parameter for a first-hand assessment of the patient's overall pulmonary state.

Thus, it was known that the shape of the capnographic curve is related to disease states of the patient. However, prior to the present invention it was entirely unexpected that the shape of the capnographic curve could also be used to estimate mismatch values between EtCO₂ and arterial PaCO₂. Just how unexpected this finding of the present inventors was, is shown by the fact that the above-mentioned EP 2 438 855 A2 in a second aspect also set out to provide a method for determining arterial PaCO₂ values. EP 2 438 855 A2 states in paragraph that “the need to be able to determine the true value of PaCO₂ from the measured value of EtCO₂ is of great importance”. In the same paragraph it is noted that the value of PaCO₂ is close to that of EtCO₂ only in subjects in good respiratory health, but that for subjects with any form of dead space ventilation, or with defective perfusion mechanisms, the two values can be widely different. The authors of EP 2 438 855 A2 attempted to solve this problem by measuring the partial pressure of oxygen in the patient's breath in addition to and simultaneously with CO₂. The obtained oxygen level is then used as an indication of the perfusion efficiency to provide an indication of discrepancy between the values of EtCO₂ obtained from the CO₂ capnographic values, and the value of the arterial PaCO₂ (EP 2 438 855 A2, paragraph [0011]). In addition to the determination of oxygen utilization, EP 2 438 855 A2 proposes to use a flow meter sensor to measure the ventilated volume of gas, which may additionally be used for estimating the discrepancy between the measured values of EtCO₂ and PaCO₂.

It is important to point out that the authors of EP 2 438 855 A2 did not consider using the shape of the capnographic curve to estimate the mismatch between the EtCO₂ and the PaCO₂ values. This is despite the fact that, according to the first aspect described in EP 2 438 855 A2, the authors knew that the shape of the curve is altered in sick individuals. The fact that the authors nevertheless chose the much more costly and elaborate methods of measuring the O₂ concentration in the breath as well as the ventilated volume of gas in addition to CO₂ shows just how surprising it was that the capnographic waveform itself could be used to correct the EtCO₂ value.

The finding by the present inventors, that the shape of the capnographic waveform itself can be used to estimate arterial PaCO₂ from respiratory EtCO₂, now makes it possible to dispense with costly additional measurements and sensors, such as for measuring O₂ and ventilated volume. Instead, the invention uses data that are already recorded in a normal capnographic measurement. Of course, the further sensors can be used in addition thereto, if an even more precise estimation of PaCO₂ is desired.

In the context of the inventive method, the CO₂ concentration levels in exhaled breath from the subject are preferably measured using a capnography sensor, preferably using the apparatus according to the invention. Advantageously, the inventive apparatus may be a portable and/or even a handheld capnograph.

The capnography signal that is obtained from the measuring of CO₂ concentration levels in exhaled breath typically is a capnogram. The capnography signal therefore preferably comprises CO₂ concentration levels as a function of time.

In an alternative embodiment of the invention, volumetric capnography may be used. In this case, the capnography signal preferably comprises CO₂ concentration levels as a function of exhaled volume. Expressing CO₂ concentration levels as function of exhaled volume can in certain cases allow even more precise estimation of mismatch and thus PaCO₂ levels.

In an embodiment, the CO₂ concentration levels are measured over a period of time corresponding to at least one breathing cycle, preferably at least two breathing cycles, more preferably at least five breathing cycles, most preferably at least ten breathing cycles. Extending the measurement over multiple breathing cycles allows to increase accuracy and precision since the influence of variations between individual breaths can be reduced. It is therefore advantageous when multiple repeated breathing cycles are measured. In the context of the invention, the term “breathing cycle” preferably corresponds to one full cycle consisting of one expiration and one inspiration.

It is further preferred if the CO₂ concentration levels are measured over a period of time of at least 5 seconds, preferably at least 10 seconds, more preferred at least 20 seconds, even more preferred at least 30 seconds, yet even more preferred at least 45 seconds, most preferred at least 60 seconds. On the other hand, the inventive method advantageously allows to obtain accurate and precise measurements in a short measurement time. Therefore, it is preferred if the CO₂ concentration levels are measured over a period of time of not more than 5 minutes, preferably not more than 3 minutes, more preferred not more than 2 minutes, even more preferred not more than 90 seconds. Preferably, the CO₂ concentration levels are measured over a period of time of between 5 seconds and 15 minutes, preferably between 10 seconds and 10 minutes, more preferred between 20 seconds and 6 minutes, even more preferred between 30 seconds and 4 minutes, most preferred between 45 seconds and 2 minutes. Extending the measurement over such a period of time allows to increase the accuracy and precision of the measurement, for the same reasons as laid out above.

In an embodiment, the measuring of CO₂ concentrations is continued until a pre-determined reproducibility criterion is met. For instance, the measurement may be repeated until a certain number of successive measurements lie within a pre-determined window of tolerance.

In a preferred embodiment, the capnography signal contains at least one, preferably at least two, even more preferred at least five CO₂ concentration values per second. It is further preferred if the capnography signal contains at least 10, preferably at least 20, more preferably at least 40, even more preferably at least 100 CO₂ concentration values. This allows even more precise and accurate measurements.

In the context of the inventive method, the deriving of the CO₂ waveform signal may consist of extracting a pre-determined time-period from the capnography signal. For instance, the CO₂ waveform signal may correspond to a certain time interval from the capnography signal, e.g. a time interval corresponding to one full breathing cycle.

The CO₂ waveform signal may correspond to the CO₂ concentration as a function of time. In an alternative embodiment, the CO₂ waveform signal corresponds to the CO₂ concentration as a function of exhaled volume. Expressing CO₂ concentration levels as function of exhaled volume can in certain cases allow even more precise estimation of mismatch and thus PaCO₂ levels.

Preferably, the CO₂ waveform signal comprises at least one expiratory upstroke and one alveolar plateau. It was found that the shape of the waveform in the expiratory upstroke and the alveolar plateau is particularly informative with regards to the mismatch.

In an embodiment, the CO₂ waveform signal further comprises at least one respiratory baseline. This provides even more information to the estimation of the mismatch and thus allows for even more accurate estimates.

Optionally, the CO₂ waveform signal may further comprise an inspiratory downstroke (alternatively or in addition to the respiratory baseline). However, for the purposes of the inventive method it is also sufficient to consider the capnographic curve only until it reaches the end-tidal value (i.e., EtCO₂).

In an embodiment, deriving the CO₂ waveform signal from the capnography signal comprises detecting individual breaths in the capnography signal. Detecting individual breaths may, e.g., be done by generating a mean line within the capnography signal corresponding to a mean CO₂ concentration (or another CO₂ concentration between the maximum and the minimum) and locating the intersections of the capnography signal with the mean line. The intersections then correspond to the transitions from inhaling to exhaling and vice versa.

In an embodiment, the CO₂ waveform signal may be extracted from one detected breathing cycle in the capnography signal. Thus, the CO₂ waveform signal may correspond to the expiratory upstroke and the alveolar plateau (and, optionally, the respiratory baseline and/or the inspiratory downstroke) of a single breathing cycle.

In a further embodiment, the CO₂ waveform signal may be extracted from multiple breathing cycles in the capnography signal. In a preferred embodiment, the CO₂ waveform signal is extracted from at least two, preferably at least three, more preferred at least six, especially at least eight breathing cycles. Preferably, the CO₂ waveform signal corresponds to the average of at least two, preferably at least three, more preferred at least six, especially at least eight breathing cycles.

Obtaining the CO₂ waveform signal as the average over multiple breathing cycles can be achieved, e.g., as follows: The individual breathing cycles can be detected as described above. Next, the capnography signal can be subdivided into the individual consecutive breathing cycles. These individual breathing cycles can then be assembled into a master breathing curve by synchronizing them (e.g., by finding a synchronization point in which the concentration change from a point to the next for the first time exceeds a certain value) followed by averaging. From this master breathing curve, the CO₂ waveform signal may then be extracted, e.g., again comprising an expiratory upstroke and an alveolar phase and, optionally, a respiratory baseline and/or an inspiratory downstroke. In this case, the expiratory upstroke of the CO₂ waveform signal corresponds to the average of the expiratory upstrokes of multiple breathing cycles, the alveolar plateau of the CO₂ waveform signal corresponds to the average of the alveolar plateaus of multiple breathing cycles, etc. Taking the average over multiple breathing cycles allows to reduce the impact of variations between individual breath and thus improves precision and accuracy.

In a preferred embodiment, the deriving the CO₂ waveform signal comprises filtering out breathing cycles that do not fulfil at least one predetermined inclusion criterion. For instance, when the capnography signal comprises at least six breathing cycles, the breathing cycle having the largest average deviation from the average (the master breathing curve) may be filtered out. In this case, the CO₂ waveform signal may correspond to the average of the remaining breathing cycles.

Methods for determining the EtCO₂ value are known in the art, as this value is commonly measured with existing capnographs. In the context of the inventive method, EtCO₂ may simply be obtained be determining the maximum CO₂ concentration of the capnography signal and/or the maximum CO₂ concentration of the CO₂ waveform signal. Alternatively, EtCO₂ may be obtained by determining the CO₂ concentration at the end of a detected expiration. As a further alternative, the CO₂ waveform signal may also be fitted to a set of mathematical functions and the EtCO₂ value may be derived from these functions.

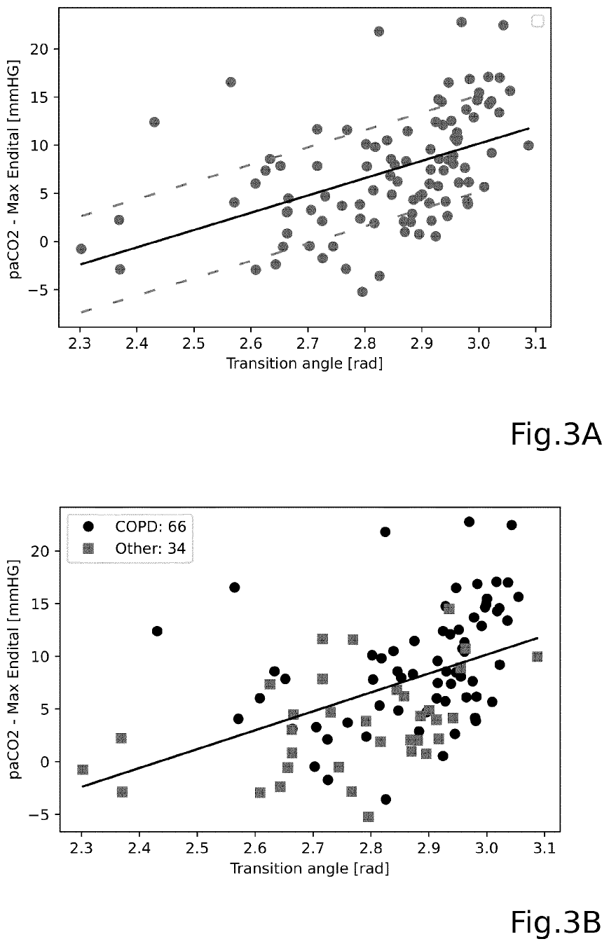

In the context of the invention, any suitable method for determining the CO₂ mismatch correction factor based on the shape of the CO₂ waveform signal may be used. In a preferred embodiment, the CO₂ mismatch correction factor is based on an angle (α) between an expiratory upstroke and an alveolar plateau of the CO₂ waveform signal. In the context of the invention, it has been surprisingly found that this angle α strongly correlates with the mismatch between respiratory EtCO₂ and arterial PaCO₂ (see Example 2 and FIG. 3). Thus, by correcting the measured EtCO₂ using the angle α, a particularly accurate estimate of PaCO₂ can be obtained.

In a preferred embodiment, the analyzing of a shape of the CO₂ waveform signal comprises fitting an expiratory upstroke to a first linear function, fitting an alveolar plateau to a second linear function and determining an angle (α) between the first linear function and the second linear function. The angle α can then be used to accurately estimate PaCO₂ based on measured EtCO₂.

In a further preferred embodiment, analyzing a shape of the CO₂ waveform signal comprises fitting a transition between an expiratory upstroke and an alveolar plateau to an exponential curve, wherein the CO₂ mismatch correction factor is based on a time constant (τ) of the exponential curve. In this case, for example, the transition between the expiratory upstroke and the alveolar plateau may be fitted to the following function:

$${p\_CO}_{2} ( t ) = {p\_CO}_{2} , {}_{∞} ( 1 - {e}^{- ( t / τ )} ) - {p\_CO}_{2} ( t = 0.1 )$$

Based on this fit the time constant τ can be determined and used for estimating PaCO₂ based on EtCO₂.

As another example, an artificial intelligence (AI) model may be used to determine the CO₂ mismatch correction factor based on the shape of the CO₂ waveform signal. In this case, the AI model may be trained using a dataset comprising both capnographic and direct blood gas measurements from healthy and/or sick individuals.

In the context of the invention the subject preferably is a human subject. In a preferred embodiment, the subject is an asthma patient and/or a Chronic Obstructive Pulmonary Disease (COPD) patient. It is further preferred, if the subject is a patient having pulmonary fibrosis. Such patients typically have increased mismatch between EtCO₂ and PaCO₂. It was surprisingly found that also in such sick patients with large mismatch values there is a strong correlation between the shape of the CO₂ waveform signal and the mismatch (see Example 2 and FIG. 3B). Thus, the present invention advantageously allows to accurately estimate PaCO₂ levels in such patients.

In many cases it can be informative to monitor the mismatch between the arterial PaCO₂ value and the respiratory EtCO₂ over a certain period of time. An increase or a decrease in the mismatch value can be indicative of a change in lung function. This is of particular interest in patients, e.g., suffering from asthma, COPD and/or pulmonary fibrosis, in whom a change of the mismatch value over time can be a critical parameter for monitoring disease progression. Thus, in a preferred embodiment, the inventive method is for monitoring mismatch values in the subject, wherein the determination of the PaCO₂ level is performed at least 2 times, preferably at least 4 times, more preferably at least 6 times, even more preferably at least 10 times over a time period of at least 1 week, preferably at least 2 weeks, more preferably at least 4 weeks, most preferably at least 8 weeks.

In the context of the inventive method, the measuring of CO₂ concentrations in exhaled breath can be done using any suitable method known to the skilled person. Advantageously, an infrared (IR) measurement method may be used. Such methods can provide reliable measurements of CO₂ concentrations. Thus, in a preferred embodiment of the inventive apparatus, the capnography sensor is an IR-based capnography sensor.

In an embodiment, the apparatus comprises a sample chamber for taking up exhaled breath from the subject. Preferably, the sample chamber is temperature-controlled. Providing a temperature-controlled sample chamber allows to reduce condensation and, in this way, increases measurement accuracy.

In an embodiment, the apparatus further comprises a flow sensor. The flow sensor may be configured to measure the volume of exhaled breath passing through the sample chamber. Providing such a flow sensor allows to record the capnography signal as a function of exhaled volume. In this case, the measured CO₂ concentrations can be scaled to the measured breath volume of the flow sensor. Thus, this embodiment is particularly preferred when the capnography signal and/or the CO₂ waveform signal corresponds to the CO₂ concentration as a function of exhaled volume. However, the data obtained from a flow sensor can also improve measurement accuracy when time capnography is used, i.e., when the CO₂ waveform signal corresponds to the CO₂ concentration as a function of time. For instance, recording the flow allows to filter out breathing cycles with insufficient flow rates, thus further improving measurement accuracy. Any suitable type of flow sensor may be used, e.g., flow sensors based on propellors, anemometer, differential pressure, or ultrasound.

Optionally, the inventive method can also comprise the measurement of O₂ partial pressure in the exhaled breath. O₂ levels may, e.g., be determined using fluorescence-based sensors. Thus, the inventive apparatus may also include an O₂ concentration sensor, preferably a fluorescence-based O₂ sensor. Measuring O₂ concentrations allows to gain additional information about lung function and serves for extended diagnostic analysis.

However, in contrast to methods disclosed in the state of art, O₂ measurements or measurements of other gases are not required for determining PaCO₂ levels. Thus, in a preferred embodiment, the inventive method does not comprise determining the concentration levels of other gases in exhaled breath, especially wherein the method does not comprise determining oxygen concentration levels. Similarly, it is preferred that the inventive apparatus does not comprise further sensors for determining the concentration levels of other gases in exhaled breath, especially sensors for determining oxygen concentration levels.

In a preferred embodiment, the sample chamber comprises a hydrophilic or a hydrophobic coating. Such coatings allow reducing interferences and can further increase measurement accuracy.

Preferably, the apparatus comprises a temperature sensor and/or a humidity sensor configured to measure the temperature and/or humidity of the exhaled breath. Preferably these sensors are located in the sample chamber of the apparatus. Such sensors allow making corrections with respect to differences in vapor partial pressure, which can influence the accuracy of measurements in certain circumstances. Thus, the inventive method preferably comprises measuring the temperature and/or humidity in the exhaled breath, in order to correct the measured CO₂ concentration levels. Similarly, the processor of the inventive apparatus is preferably configured to correct the CO₂ concentration levels obtained from the capnography sensor based on the measured temperature and/or humidity.

In a further embodiment, the apparatus comprises a dichroic or semitransparent mirror, preferably in the sample chamber. Such a mirror allows detecting condensation and other interference optically.

In a preferred embodiment, the apparatus further comprises a mouth piece. A mouth piece allows to deliver breath to the apparatus in a particularly convenient manner. Alternatively, the breath may also be delivered to the apparatus through a breathing mask or through a nasal probe. Advantageously, the mouth piece, the breathing mask and/or the nasal probe may comprise an antimicrobial, especially an antibacterial and/or antiviral, coating and/or filter.

In a preferred embodiment of the inventive method, the CO₂ concentration levels in exhaled breath is measured using a mainstream capnograph. Similarly, it is preferred if the inventive apparatus is a mainstream capnograph. By using a mainstream capnograph, volume losses can be avoided and the whole respiratory gas volume can be measured. This allows particularly precise determination of PaCO₂ levels using the inventive method. Mainstream capnographs are preferred over so-called sidestream capnographs, which are typically used in ventilated patients and in which only a respiratory gas sample from the airway opening of the ventilated patient is analyzed.

In the context of the inventive method, the subject may be in any state. However, the inventive method is particularly advantageous when the subject is in a conscious state. Preferably, the subject is in an awake state. Preferably, the subject is not artificially ventilated.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Unless specified otherwise, the term “CO₂ concentration levels” or similar terms as used herein refers to the partial pressure of CO₂.

Unless specified otherwise, all parameters as used herein correspond to parameters at a temperature of 33° C. and a pressure of 101.300 Pa.

The present invention is further illustrated by the following figures and examples, without being limited thereto.

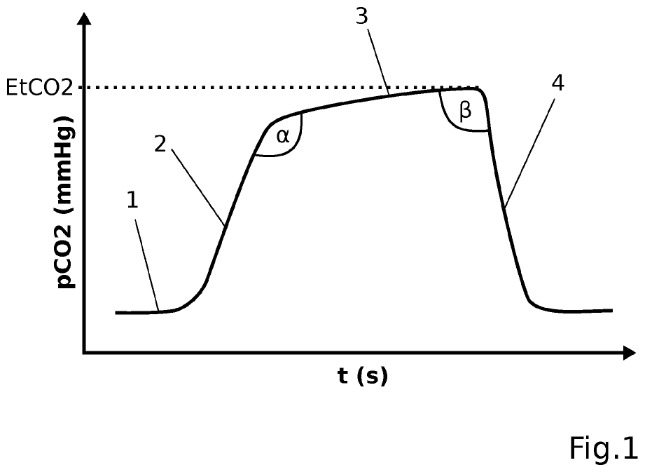

FIG. 1. Schematic depiction of a CO₂ waveform signal corresponding to one respiration cycle of a capnographic signal. The graph displays CO₂ partial pressure (pCO2; e.g., measured in millimeter of mercury, mmHg) on the y-axis and time (t; e.g., measured in seconds, s) on the x-axis. The depicted CO₂ waveform signal comprises a respiratory baseline 1, an expiratory upstroke 2 (also referred to as alveolar rise), an alveolar plateau 3, and an inspiratory downstroke 4. Also displayed are the angle α between the expiratory upstroke 2 and the alveolar plateau 3, as well as the angle β between the alveolar plateau 3 and the inspiratory downstroke 4. The end-tidal carbon dioxide (EtCO₂) is displayed as the maximum CO₂ concentration of the CO₂ waveform signal.

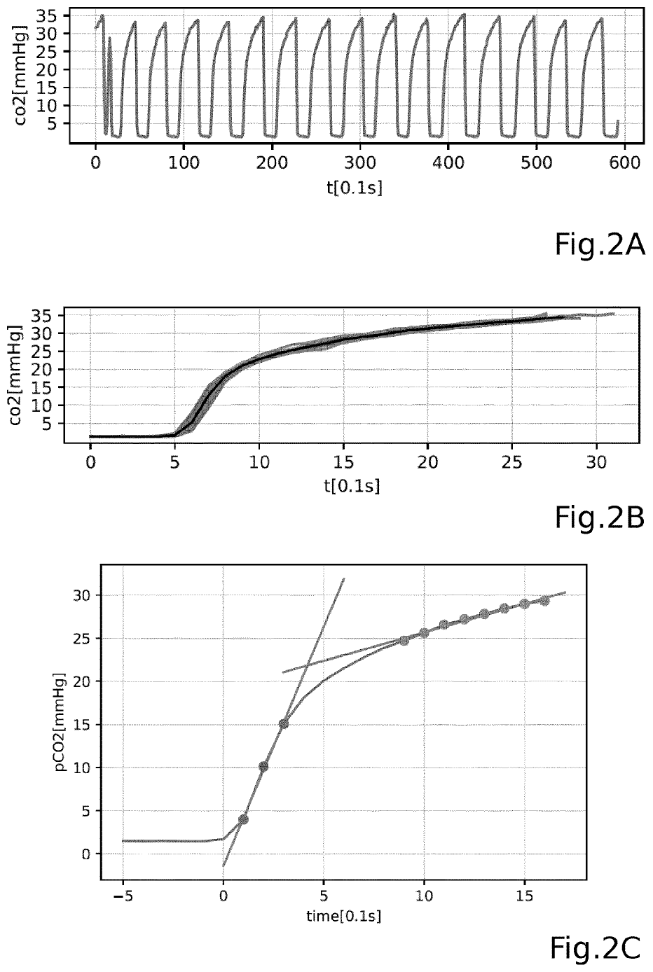

FIG. 2. Determination of a PaCO₂ level according to an exemplary embodiment of the present invention. (A) Capnography signal obtained from a human adult subject. (B) Overlay of sections from the capnography signal corresponding to 14 synchronized breathing cycles extracted from the capnography signal. (C) CO₂ waveform signal corresponding to the mean of the overlaid curves shown in panel A. The expiratory upstroke and the alveolar plateau were each fitted to a linear function and the angle α between these lines was calculated.

FIG. 3. Results from a correlation study involving 34 healthy subjects and 66 patients diagnosed with COPD. The mismatch between PaCO₂ determined from blood samples and EtCO₂ determined by capnography is displayed on the y-axis. The angle α obtained from the capnography measurements is displayed on the x-axis. (A) Graph including all subjects of the correlation study. (B) Same graph, wherein healthy subjects (“Other”) and COPD patients are displayed individually.

### EXAMPLE 1. DETERMINATION OF A PACO₂ LEVEL IN A SUBJECT

The present example describes the determination of a PaCO₂ level in a subject according to an exemplary embodiment of the invention.

### Recording of Capnography Signal

A capnography signal was obtained from a human adult subject using a capnograph. Specifically, a main-stream capnograph with a nondispersive infrared spectrometer having a CO₂ sensor with a wavelength of 4.26 micrometers and a reference sensor at 3.91 micrometers was used. The subject was allowed to breathe through the mouthpiece of the capnograph for a time period of 1 minute. The CO₂ content in the breath was determined by analyzing the absorption of light in the CO₂ oscillation spectrum compared to calibration data. The CO₂ concentration (CO₂ partial pressure in mmHg) was recorded with a time resolution of 10 Hz, corresponding to 600 measurements per minute. The obtained capnography signal is shown in FIG. 2A.

### Detection of Breathing Cycles

For detecting individual breaths in the obtained capnography signal, a mean line was generated, corresponding to the mean CO₂ concentration in the capnography signal. The intersections were then taken as the transitions from inhaling to exhaling and vice versa. In total, 14 breathing cycles were detected.

### Generation of a Master Breathing Curve

The capnography signal was separated into 14 sections corresponding to the individual breathing cycles detected as described above. For each section, an EtCO₂ value corresponding to the maximum detected CO₂ concentration was determined. In the context of the present example, only the data up to the EtCO₂ were used; thus, the data points following the EtCO₂ value were deleted from each section.

Next, a synchronization point within each section was identified as the first data point in which the CO₂ concentration change with respect to the previous data point exceeded 1 mmHg. The sections were then synchronized based on the synchronization point and overlayed. The resulting overlay of the 14 synchronized sections is shown in FIG. 2B.

In order to obtain a master breathing curve, the mean of the 14 synchronized sections was taken (i.e., at each time point the mean of the 14 CO₂ concentrations was calculated). The resulting master breathing curve was used as the CO₂ waveform signal for further analysis.

Deriving Parameters from the CO₂ Waveform Signal

The EtCO₂ value was obtained by determining the maximum CO₂ concentration of each of the 14 sections used to obtain the master breathing curve and calculating the average of the 14 maxima obtained.

Next, the shape of the CO₂ waveform signal was analyzed. In the present example, the angle α between the expiratory upstroke 2 and the alveolar plateau 3 was used as a characteristic parameter of the shape in order to determine a CO₂ mismatch correction factor. To determine the angle, the datapoints of the CO₂ waveform signal corresponding to the expiratory upstroke 2 and the alveolar plateau 3 were determined. Next, the datapoints of the two sections were fitted to two linear functions and the angle between the two linear functions was calculated. This is shown in FIG. 2C.

### Calculation of PaCO₂

The PaCO₂ level was calculated by applying the CO₂ mismatch correction factor based on the angle α to the obtained EtCO₂ value. For this purpose, the linear correlation between α and mismatch shown in Example 2 was used.

More specifically, the data in Example 2 were fitted to a function MM=m*α+b using linear regression, MM being the mismatch value (PaCO₂−EtCO₂). For determining the mismatch values from the actual measurement, the same formula was used by inserting the values obtained for the slope m and the intercept b, as well as the measured angle. PaCO₂ was then calculated by adding the mismatch value to EtCO₂ (PaCO₂=EtCO₂+MM).

### EXAMPLE 2. CORRELATION BETWEEN MISMATCH AND CAPNOGRAM SHAPE

In order to investigate the correlation between the mismatch and parameters relating to the capnogram shape, capnograms of 100 subjects were recorded and analyzed, essentially as described in Example 1. The study included 34 healthy subjects and 66 patients diagnosed with COPD.

In addition to the capnographic measurements, blood from each subject was collected and the CO₂ concentration was analyzed by blood gas analysis. The mismatch between EtCO₂ determined by capnography and the PaCO₂ determined from the blood samples was calculated. FIG. 3A shows the correlation between the mismatch and the angle α between the expiratory upstroke 2 and the alveolar plateau 3. As can be seen from the figure, a strong correlation was observed.

FIG. 3B shows the same correlation, wherein the datapoints from healthy subjects (“other”) and the COPD patients are identified. As can be seen from this figure, in both cases a strong correlation between the angle α and the mismatch was observed.

### EXAMPLE 3. VALIDATION WITH HUMAN SUBJECTS

A validation study was carried out with 101 human subjects, 67 of whom were COPD patients. For each subject, PaCO₂ levels were determined by capnography and analyzed by the inventive method as described in Example 1. For comparison, the data obtained from the capnographic measurement were used without mismatch correction, by simply determining the EtCO₂ level. In addition, immediately following the capnographic measurement, blood was drawn from the subjects' earlobes to obtain capillary blood samples for blood gas analysis. The CO₂ levels obtained using capnography with or without the inventive method as well as using blood gas analysis were then compared. The following results were obtained:
Capnographic measurement without mismatch correction (EtCO₂): approx. 28% of the measurements were within +/−5 mmHg of the results obtained from blood gas analysis;
Capnographic measurement according to the invention (including mismatch correction as described in Example 1): approx. 71% of the measurements were within +/−5 mmHg of the results obtained from blood gas analysis.

Thus, as can be seen from these results, the PaCO₂ values obtained using the inventive method were much closer to the values determined by blood gas analysis than when only the EtCO₂ values were used (as is done with common capnometers or capnographs). The improvement in the accuracy was observed both for healthy subjects and COPD patients, but was particularly pronounced with sick patients.

### EXAMPLE 4. CORRECTION BASED ON FURTHER PARAMETERS CHARACTERIZING THE SHAPE OF THE CO₂ WAVEFORM SIGNAL

A master breathing curve was obtained as described in Example 1 and used as the CO₂ waveform signal for further analysis. Also, the EtCO₂ value was determined in the same way as described in Example 1. However, an alternative approach was used to analyze the shape of the CO₂ waveform signal.

First, the CO₂ waveform signal was fitted using cubic splines. The CO₂ waveform signal was described by the following mathematical function:

$$f ( t ; {c}_{1} , {c}_{2} , τ , k ) = {c}_{2} - ( {c}_{2} - {c}_{1} ) {e}^{- \frac{t - {t}_{0}}{τ}} + k ( t - {t}_{0} )$$

$$\frac{d}{dt} f ( t ; {c}_{1} , {c}_{2} , τ , k ) = \frac{( {c}_{2} - {c}_{1} )}{τ} {e}^{- \frac{t - {t}_{0}}{τ}} + k$$

The parameters c₁ and c₂ are given with:

$${c}_{2} = {c}_{1} + τ ( {f}^{′} ( {t}_{0} ) - k )$$

$$f ( {t}_{0} ) = {c}_{1}$$

In the above formulas, t is the time, t₀ is time at the inflection point marking the start of the expiratory upstroke, k the constant of the linear term, and τ is the time constant of the exponential term. The parameters are determined by fitting this mathematical function to the Mastercurve beginning at t₀.

The parameter τ describes how stretched or compressed the CO₂ waveform signal is. The mismatch correction was carried out using this parameter τ and the linear term k.

### Calculation of PaCO₂

The PaCO₂ level was calculated by applying the CO₂ mismatch correction factor based on the parameters τ and k to the obtained EtCO₂ value. For this purpose, a linear correlation between those factors and mismatch analog to that shown in Example 2 was used.

More specifically, the data in Example 4 were fitted to a function MM=m₁*+τ+m₂*k+b using linear regression, MM being the mismatch value (PaCO₂−EtCO₂). For determining the mismatch values from the actual measurement, the same formula was used by inserting the values obtained for the slopes m₁ and m₂ and the intercept b, as well as the factors τ and k. PaCO₂ was then calculated by adding the mismatch value to EtCO₂ (PaCO₂=EtCO₂+MM).

The data from the validation study in Example 3 were analyzed using the above mismatch correction instead of using the angle α. The following results were obtained:
Capnographic measurement without mismatch correction (EtCO₂): approx. 28% of the measurements were within +/−5 mmHg of the results obtained from blood gas analysis;
Capnographic measurement according to the invention (including mismatch correction as described in Example 1): approx. 79% of the measurements were within +/−5 mmHg of the results obtained from blood gas analysis.

Thus, the above-described method for analyzing the shape of the CO₂ waveform signal led to ever higher accuracy than the analysis based on the angle α.

1. A method for determining an arterial CO₂ concentration (PaCO₂) level in a subject, the method comprising the following steps:
measuring CO₂ concentration levels in exhaled breath from the subject over a period of time in order to obtain a capnography signal;
deriving a CO₂ waveform signal from the capnography signal;
determining an End-tidal CO₂ (EtCO₂) value from the CO₂ waveform signal;
analyzing a shape of the CO₂ waveform signal in order to determine a CO₂ mismatch correction factor;
applying the CO₂ mismatch correction factor to the EtCO₂ value in order to obtain the PaCO₂ level.

2. The method according to claim 1, wherein the CO₂ waveform signal comprises at least one expiratory upstroke and one alveolar plateau, wherein the CO₂ mismatch correction factor is based on an angle between the expiratory upstroke and the alveolar plateau.

3. The method according to claim 1, wherein the CO₂ waveform signal comprises at least one expiratory upstroke and one alveolar plateau (3), wherein analyzing a shape of the CO₂ waveform signal comprises fitting a transition between the expiratory upstroke and the alveolar plateau to an exponential curve, wherein the CO₂ mismatch correction factor is based on a time constant of the exponential curve.

4. The method according to claim 1, wherein the CO₂ waveform signal corresponds to the CO₂ concentration as a function of time.

5. The method according to claim 1, wherein the CO₂ waveform signal corresponds to the CO₂ concentration as a function of exhaled volume.

6. The method according to claim 1, wherein the CO₂ concentration levels are measured over a period of time of between 10 seconds and 5 minutes.

7. The method according to claim 1, wherein the CO₂ waveform signal corresponds to the average of at least two breathing cycles.

8. The method according to claim 1, wherein the method does not comprise determining the concentration levels of other gases in exhaled breath, especially wherein the method does not comprise determining oxygen concentration levels.

9. The method according to claim 1, wherein the subject is an asthma patient, a Chronic Obstructive Pulmonary Disease (COPD) patient, and/or a pulmonary fibrosis patient.

10. The method according to claim 1, wherein the method is for monitoring mismatch values in the subject, wherein the determination of the PaCO₂ level is performed at least 4 times over a period of time of at least 2 weeks.

11. An apparatus for determining a PaCO₂ concentration level in a subject, the apparatus comprising:
at least one capnography sensor configured to measure CO₂ concentration levels in exhaled breath from the subject as a function of time;
a processor configured to carry out the following steps:
obtaining from the capnography sensor a capnography signal comprising CO₂ concentration levels in exhaled breath from the subject over a period of time;
deriving a CO₂ waveform signal from the capnography signal;
determining an End-tidal CO₂ (EtCO₂) value from the CO₂ waveform signal; analyzing a shape of the CO₂ waveform signal in order to determine a CO₂ mismatch correction factor;
applying the CO₂ mismatch correction factor to the EtCO₂ value in order to obtain the PaCO₂ level.

12. The apparatus according to claim 11, wherein the apparatus further comprises a flow sensor.

13. The apparatus according to claim 12, wherein the apparatus further comprises a sample chamber for taking up exhaled breath from the subject, wherein the sample chamber is temperature-controlled.

14. The apparatus according to claim 12, wherein the apparatus comprises a temperature sensor and/or a humidity sensor and wherein the processor is configured to correct the CO₂ concentration levels obtained from the capnography sensor based on the measured temperature and/or humidity.

15. The apparatus according to claim 12, wherein the processor is configured to carry out the following steps:
measuring CO₂ concentration levels in exhaled breath from the subject over a period of time in order to obtain a capnography signal;
deriving a CO₂ waveform signal from the capnography signal;
determining an End-tidal CO₂ (EtCO₂) value from the CO₂ waveform signal;
analyzing a shape of the CO₂ waveform signal in order to determine a CO₂ mismatch correction factor;
applying the CO₂ mismatch correction factor to the EtCO₂ value in order to obtain the PaCO₂ level.
